(19) United States
(12) Patent Application Publication
(10) Pub. No.: US 2013/0310657 A1
(43) Pub. Date: Nov. 21, 2013

(54) Electronic Monitoring System for Data Collection, Presentation and Analysis

(71) Applicants: Colin Edward Sullivan, Balmain (AU); Peter Charles Spencer, Coogee (AU)

(72) Inventors: Colin Edward Sullivan, Balmain (AU); Peter Charles Spencer, Coogee (AU)

(73) Assignee: SONOMEDICAL PTY LTD, Balmain, New South Wales (AU)

(21) Appl. No.: 13/825,848

(22) PCT Filed: Sep. 24, 2011
(86) PCT No.: PCT/AU2011/001227

§ 371 Date: Jul. 22, 2013

(30) Foreign Application Priority Data
Sep. 24, 2010 (AU) 2010904316

(51) Int. Cl. A61B 5/00; A61B 7/00

(52) U.S. Cl. CPC A61B 5/7455; A61B 5/0002; A61B 7/00; A61B 5/7405; A61B 5/742
USPC 600/301

(57) ABSTRACT

An assembly for monitoring physiological parameters of a living subject at a sensing station, the assembly comprising: a plurality of sensors arranged about the subject to create at least one sensor field in which said sensors are capable of sensing one or more said parameters of the subject in each said at least one sensor fields; electronic means to transmit data sensed by each said sensors of said at lest one sensor field to at least one data receiving station; a controller for controlling transmission of said data from said sensor fields to said receiving station; means for an operator to optimise signals received from the sensor fields; wherein the data includes sensed movements and sounds within the range of 0.5 Hz-20000 Hz; and wherein data obtained from the sensor fields includes data capable of interpretation by the use of the sense of touch.

### BACKGROUND SUMMARY

The present invention relates to methods and apparatuses for electronic monitoring of patient parameters for medical diagnostic purposes and a variety of other purposes. The invention also relates to contact sensing and wireless monitoring of such parameters and optimising of signal data obtained. The invention also relates to a monitoring assembly and apparatus which employs sensors forming one or more sensor fields which are either in contact with or not in contact with a subject, the latter to enable wireless monitoring and measurement of parameters over a predetermined period of time related, but not limited to, such actions as breathing, heartbeat, body movements, body position, gut activity, jaw movements, sleep performance including snoring, sleep apnea, restricted airways, asthma, quiescent periods, period spent asleep or awake and fetal heart beat, fetal movements, placental blood flow and the like. The Invention further relates to an apparatus which allows data obtained from a sensor field to be interpreted using the sense of touch to thereby simulate physical examination of a subject.

### PRIOR ART

Sounds generated by physiological activity have long been monitored by physicians as an important source of diagnostic information. The sounds generated by physiological activity arise from a number of sources such as: breathing, heart activity, blood flow in various blood vessels and organs, digestion and skeletal muscle activation and joint movement vibrations. Many other of the body functions generate sounds, or sub audible vibrations. Breathing sounds are generated by turbulence as air flows in and out of the lungs. Normal breathing sounds can generally be found in the frequency range of approximately 30-900 Hz but are typically of low amplitude and inaudible without the use of an amplifier (e.g. stethoscope). Snoring is often seen in the frequency range of 30-250 Hz and are typically loud and audible whereas components of wheezing can often be found over a broad frequency range form 100-2000 Hz. Breathing movements may often be seen in the 0.05 to 2 Hz range.

Examples of conditions involving abnormal breathing sounds include pneumonia, pleural rub snoring, crepitations and wheezing, the latter typical of asthma, resulting from turbulence when air flows through a narrowed part of a large airway. Crepitation ( heart failure) sounds are typical in congestive heart failure reflecting the occurrence of small airway closure and opening due to presence of fluid in the lung. Normal heart sounds are generated by closing of the four heart valves, and abnormal sounds (murmurs) occur with turbulence across the valves.

Abnormal gut sounds may arise from the reflux of fluid from the stomach into the esophagus and or increased peristalsis. Listening to such sounds plays a central role in the physical examination of a subject and many diseases and abnormalities are identified in part by the hearing of abnormal sounds. A device which has been used by medical staff to detect both normal and abnormal sounds is the stethoscope. This instrument is a physical amplifier, which enables a medical attendant to hear very low volume sounds, and is a principal tool of the physician.

Electronic monitoring systems have been proposed for medical use, which attempt to emulate a physician with a stethoscope by both listening and analysing the sounds heard. Sensitive transducers perform the listening function and they typically produce a signal in which the sound signals are aliased. Algorithms can then be applied to the signal to attempt to interpret the sounds heard. Typical electronic stethoscope devices require the sensor to be manually held against the subjects torso in order to enable their function.

The present applicant's own application PCT/AU2002/00615 discloses an electronic monitoring system for medical use, which uses transducers to record at least one channel of sound information from a subject and display a visual trace representing a channel of sound information relative to time. That invention also employs an audio system to selectively replay sound information recorded from a subject and displayed by a visual trace and a cursor which travels over the visual trace as the sound information is replayed. This enables identification of an instant on the visual trace that corresponds to the sound heard at the same instant Each transducer may produce more than one channel of output, for instance for different frequencies ranges or intensities of sound. Several of the transducers may be used to record different channels of sound information at the same time from different locations on the patient. Several visual traces relating to different recorded signals may be displayed at the same time. Traces may represent physiological parameters such as respiratory airflow, or electrocardiogram or pulse etc. A variety of visual traces can be selected. It is then possible to replay the sound for that selected section of the trace. The cursor continually tracks the trace, or selected section of the trace, whilst the sound information is played. In this way the medical staff are able to correlate the sound information played on the audio system with the information displayed on the visual trace.

A visual display of sound and movement recordings (which may include a range of contributing sources of sound—eg breathing sounds, heart sounds, ambient sounds) allows an end user, by using his/her visual skills to readily identify points in time where sounds of interest have occurred, then quickly and efficiently, by replaying a “sound bite” repeatedly, allow the auditory skills of the brain to identify the nature and origin of the sound. The visual display can be a voltage trace which by itself gives some idea of the signal amplitude but not the frequency breakdown. In this way the method allows an end user to see when noise occurred, and then hear and separate the components of noise of interest.

The prior art also teaches the use of an array of transducers placed within a mattress to record sound and movement of a subject. Transducers have also been placed in sleeves of a patient's garments and placed against the patient. In those circumstances the use of sensor leads connecting sensors to reading devices can tangle the patient. In the case of using this equipment with infants, the leads may be dangerous as they can present a strangulation risk. Home testing of patients using monitoring is also known and normally requires belts and leads making it cumbersome and uncomfortable for patients to manage. To date the prior art has concentrated on sleep studies using such apparatuses as belts around the abdomen, various contact sensors placed on the head, and measurements of chin muscle activity.

What that application does not teach is a technique for contactless sensing and use of optimisation of such signals obtained by the sensors to determine optimal signals for each parameter selected. Also, the prior art application docs not teach use of the sense of touch contact with a device or element which allows a simulated remote physical examination of a living subject.

Typical prior art methods for estimating whether a patient is asleep or awake include EEG and actigraphy. EEG requires attachment of leads to the subject. While actigraphy can be completed using sensors not connected to the subject it is designed to determine if a subject is moving or not and cannot accurately determine whether a non moving subject is asleep or awake and therefore often tends to overestimate a subjects sleep time. Hence currently there are no accurate methods using sensors not attached to the subject which can accurately determine if the subject is awake or asleep.

When completing and analysing a study to assess a patient's sleep, one metric required is the length of time that the patient is in bed intending to sleep. Typical prior art sensing systems use sensors attached directly to the patient. Prior art systems assume that the intention to sleep time interval is determined as the period during which the sensor systems are attached to the patient's body and switched on, irrespective of whether the patient is in bed or not. The intention to sleep period is used in estimating the subjects sleep efficiency. It is used as the denominator when determining the percentage of sleep achieved during the period that the patient was intending to sleep. Prior art systems may overestimate the time a subject is intending to sleep if the subject spends significant time doing activities other than trying to sleep while attached to the sensors .

Remote monitoring from remote sensing equipment is also known in the prior art. Patent publication PCT/AU00/00149 discloses an apparatus for physiological monitoring of a remote subject. That system employs monitoring probes which are connected to the subject. Prior art systems use several methods to assess the occurrence and severity of physiological events and pathologies including hypopnea, obstructed breathing, snoring, cough, wheeze, crepitation, tooth grinding, vomiting, body movements such as myoclonus and esophageal reflux, Prior art methods include use of a stethoscope, thermistor and nasal flow pressure breath flow rate estimates, esophageal manometry to measure flow resistance, esophageal reflux catheter to measure reflux severity, actigraphy, EMG, EEG, oximetry and microphone. Event severity in prior art systems is most often measured qualitatively or quantified as event frequency per unit time without reference to the magnitude of each individual event occurrence or the cumulative magnitude of all events over time. Additionally in the case of oximetry and or carbon dioxide detection they are used as a consequentially related measure of air flow obstruction severity. In the case of infants although it is generally recognized that labored breathing due to increased air flow resistance is potentially damaging independent of the occurrence of apnea or major blood oxygen decreases no quantitative measure is widely used to evaluate flow restriction severity. Some researchers have studied the use of microphones attached to the subject to measure snoring intensity as an estimate of respiratory effort or inspiratory volume decrease. In this case snoring intensity was recorded on a polygraph and no evaluation was completed of the frequency characteristics of the signals (Stoohs et al: Respiration Physiology, 92, 27-38, 1993). This study did not try to nor was its aim to estimate snoring severity or intensity throughout the sleep period but rather look at time point evaluation over short periods.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### INVENTION

The present invention provides electronic monitoring of patient parameters for medical and related diagnostic purposes and more particularly provides contact sensor and wireless sensor monitoring of such parameters. The invention further provides a monitoring system and associated apparatus which employs sensors fixed to a subject, sensors not fixed to a subject or a combination of both and which sense, measure and transmit parameters over a predetermined period of time related, but not limited to, breathing, heartbeat, body movements, sleep performance including coughing, vomiting, crying, groaning, sniffing, shivering, snoring, sleep apnea, restricted airways, asthma, crepitation, heart valve movement and sounds, fibrillations, ectopic beats, tremors, rolling movements, fitting, reflux and other digestive sounds bodily fluid flows breathing effort, bruxism, fetal heartbeat, fetal movements, fetal breathing effort, placental blood flow and non diagnostic or not necessarily diagnostic environmental noise. The invention enables a subject to move freely within a wireless sensing “field” and to be continuously monitored for signals of interest while within the sensor field without in any way being impeded or disturbed by the sensing system. In this system the surface, such as a mat, in or about which wireless sensors are mounted, may be part of the sensor field. Signals can be transmitted from a subject lying against the mat into and along or within the mat and thence into one or more sensors. The invention further provides a sensing assembly which includes data capable of analysis by touch. The invention further comprises a method for optimising signals obtained from a contactless sensor field. It will be understood that sensors may be mounted on or in a range of different objects around a lying, sitting or ambulatory subject or subjects to create a sensor field.

The present invention also provides a wireless monitoring system for analysis of traces of patient parameters using algorithms to identify and measure a pathological state. Wireless in the present context can be taken to mean contactless in that the patient is not directly connected to sensors but is close enough to a sensor location to enable the sensor to detect a body parameter remotely. Body parameters which can be sensed wirelessly include but are not limited to breathing (2-3 kHz), snoring (200-900 Hz), gross body movements and other time sequenced health and breathing related sounds. Vibrational sound and movement signals are detected by the wireless sensors. Vibrational sounds can be assessed by touch to provide a simulated real time physical examination and can also be optimised to obtain the best data which can then be used to evaluate certain anatomical pathologies. Likewise data related to temperature of a subject can also be analysed by touch. The system also allows for the use of a wide variety of sensor types aand sensor field configurations including wireless electromagnetic field sensors of the type which can be used to record electroencephalograms (EEG), electrocardiogram (ECG), eye movement (EOG) and muscle activity (EMG) [sensors of the type described by Prance et al, Applied physics letters 93,033906 (2008) and Sullivan et al, www.biology.ucsd.edu (2007)], Other possible wireless sensor types include temperature, moisture or chemical (eg ammonia) sensors and force sensors which may provide information on the subjects temperature, sweating, biochemical or respiratory status and position in the sensor field

According to one embodiment contact or non contact sensors may be used to measure severity of airway flow restriction. Microphones could also be used to detect such severity.

In particular sensors placed on either side of the position where a subject's pillow would be placed is found to be advantageous in a number of ways. Microphone sensors in these positions largely remain uncovered by sheets or blankets during the recording period. In addition recording from two microphones taken from either side of the patients head provide significant information about the subjects position and orientation during sleep as sound recordings for instance will be louder on the side a subject is facing if they are sleeping in the lateral position. This orientation related information is useful for instance during assessment of sleep studies. Microphones placed in these positions will also provide useful information about noises originating from a sleeping partner or other atmospheric noise including dog barking, train, car, aircraft and thunder sounds which may have significant effects on the subjects sleep quality and should be considered during such an evaluation.

According to one embodiment the system is self tuning and allows automatic analysis of the traces including analysis of such traces using algorithms. The system identifies data periods including the length and time sequence of body movement.

It is one object of the invention to provide a non invasive and minimally intrusive system for monitoring patients. It is a further object of the invention to provide a more convenient and patient friendly method of body parameter monitoring in a home, doctors offices, nursing stations, emergency departments, ambulances and general medical or laboratory environment and which is free of the disadvantages of complex sensor and probe contact or direct engagement with the subject. The system can include a range of formats requiring only that the subject lie or sit against a surface within, on or about which the wireless sensors are mounted including mat, mattress, trolley, chair and other system formats. It is a further object of the Invention to provide long or short period monitoring at home or in a controlled environment, to increase the nature and type of pathologies which can be monitored and determined from data provided by such monitoring. The system can be used as a means of facilitating telemedicine where a subject in the sensor field can be monitored and diagnosed remotely in real time by a clinician. In this case signals from the system would be transmitted via the internet or other means from the subject's position to the clinicians consulting room for instance.

In one broad form the present invention comprises:

an assembly for monitoring physiological parameters of a living subject at a sensing station, the assembly comprising:

at least one sensor field in which sensors are capable of sensing one or more said parameters of the subject in each said at least one sensor fields;

electronic means to transmit data sensed by each said sensors of said at lest one sensor field to at least one data receiving station;

wherein the data includes sensed movements and sound within the range of 0.5 Hz-20,000 Hz; and

wherein data obtained from the sensor fields includes data capable of interpretation using the sense of touch.

Preferably the assembly includes a controller for controlling transmission of said data from said sensor fields to said at least one receiving station for processing in a processing station. Preferably, data capable of interpretation using the sense of touch includes data capable of generating vibrations in an output device such as but not limited to a touch pad, audio speakers or equivalent.

According to a preferred embodiment each signal capable of analysis by touch and feel can be adjusted to render the signal optimal for assessment and analysis. One way this can be done is to selectively adjust signal amplitude and frequency.

In an alternative embodiment the data capable of interpretation using the sense of touch includes temperature related data from a subject allowing a practitioner to feel temperature.

In another broad form the present invention comprises:

an assembly for monitoring physiological parameters of a living subject at a sensing station, the assembly comprising:

a plurality of sensors arranged about the subject to create at least one sensor field in which said sensors are capable of sensing one or more said parameters of the subject in each said at least one sensor fields;

electronic means to transmit data sensed by each said sensors of said at lest one sensor field to at least one data processing station;

a visual/readout and aural data;

means for an operator to optimise signals received from the sensor fields;

wherein the data includes sensed movements and sounds within the range of 0.5 Hz-20,000 Hz; and

wherein data obtained from the sensor fields includes data capable of interpretation by the use of the sense of touch.

According to one embodiment the sensors are not fixed in direct contact with the subject and are integrated with a device on which a subject is located tor monitoring.

In another broad form of an apparatus aspect the present invention comprises:

a system for obtaining parameter data from physiological monitoring of a subject for diagnostic purposes, the system comprising;

at least one fixed sensor and/or at least one microphone placed in a sensor field created by said at least one sensor and/or at least one microphone,

detection means to allow said at least one sensor and/or at least one microphone in the sensor field to detect breathing effort and flow, sounds and body movements of a subject free to move about the sensor field relative to the sensors and microphones so that data concerning the sleep wake cycle of the subject are readable by the sensors over a predetermined period of time irrespective of whether the subject is remote from or in transient contact with one or more said sensors,

transmitting means to display signals taken from the subject readings via the sensors and /or microphones of predetermined parameters over a predetermined time period as the subject moves relative to the at least one sensor,

selecting from the display a plurality of signals provided by said at least one sensor and optimizing the signal to enhance diagnostic capability from the available signal data identified by movement parameters; wherein at least some of the data obtained from the sensor fields can be interpreted by the sensor of touch;

reading parameter data related to movement and quiescent periods from an optimized signal and interpreting the optimized signal for diagnostic purposes and to determine periods of waking and sleeping.

In another broad form of the apparatus the present invention comprises:

an electronic monitoring system for medical use, comprising:

a plurality of fixed sensors and/or at least one microphone placed in a sensor field created by said at least one sensor and/or at least one microphone to record at least one parameter from a subject relating to a physiological condition of the subject,

detection means to allow said at least one sensor and/or at least one microphone in the sensor field to detect parameters from a subject free to move about the sensor field relative to the sensors and microphones so that body parameters are readable by the sensors over a predetermined period of time irrespective of whether the subject is remote from or in transient contact with one or more said sensors,

a readout device to display at least one visual trace representing the at least one parameter signals taken over a given time period; a cursor which travels over the visual trace as the parameter information is replayed to identify the instant on the visual trace that corresponds to the sound heard at the same instant; means to play and to enable feel of sound or vibration related information or temperature recorded from a subject and displayed by the at least one visual trace and through a touch reading device such as touch pad or thermometer; optimizing signal displays relative to a particular parameter to allow a diagnosis of an event or condition in the subject.

In one broad form of a method aspect, the present invention comprises:

a method of data collection from a living subject and analysis of said data using an assembly comprising,

a plurality of sensors;

at least one sensor field formed by at least one sensor from said plurality of sensors;

electronic means to transmit data sensed by each said sensors to at least one data processing station;

the method comprising the steps of:

locating a subject on or near a monitoring device;

taking a plurality of sensors and arranging the sensors relative to the subject to create Bt least one sensor field about the subject;

activating the sensors to enable them to sense one or more functional parameters of the subject in each said at least one sensor fields;

transmitting data measured from the sensors in data fields to a receiver thereby creating visual read outs tactile data and sounds,

optimising data signals received from the sensor fields by selecting optimal signals from the data.

wherein the data includes sensed movements and sounds within the range of 0.5 Hz-20,000 Hz; and

wherein data obtained from the sensor fields includes data capable of analysis by touch.

According to one embodiment the data capable of analysis by touch includes vibrational and temperature data.

In another broad form of a method aspect, the present invention comprises:

a method for obtaining parameter data from physiological monitoring of a subject for diagnostic purposes, the method comprising the steps of;

setting up a monitoring system including at least one fixed sensor and/or at least one microphone placed in a sensor field created by said at least one sensor and/or at least one microphone,

placing a subject in the sensor field such that the subject is free to move about the sensor field and so that body parameters are readable by the sensors over a predetermined period of time irrespective of whether the subject is remote from or in transient contact with a sensor by movement towards the sensor,

taking from the subject readings via the sensors and/or microphones of predetermined parameters over a predetermined time period as the subject moves relative to the at least one sensor;

selecting from a plurality of signals provided by said at least one sensor and optimizing the signal to enhance diagnostic capability from the available signal data identified by performance parameters;

reading parameter data from an optimized signal and interpreting the optimized signal.

The present invention provides an alternative to the known prior art and the shortcomings identified. The foregoing and other objects and advantages will appear from the description to follow. In the description reference is made to specific embodiments in which the invention may be practiced. These embodiments will be described in sufficient detail to enable those skilled in the art to practice the invention, and it is to be understood that other embodiments may be utilized and that structural changes may be made without departing from the scope of the invention. The following detailed description is, therefore, not to be taken in a limiting sense, and the scope of the present Invention is best defined by the appended claims.

### BRIEF DESCRIPTION OF THE REPRESENTATIONS

The present invention will be described in more detail below according to preferred but non limiting embodiment and with reference to the accompanying illustrations wherein:

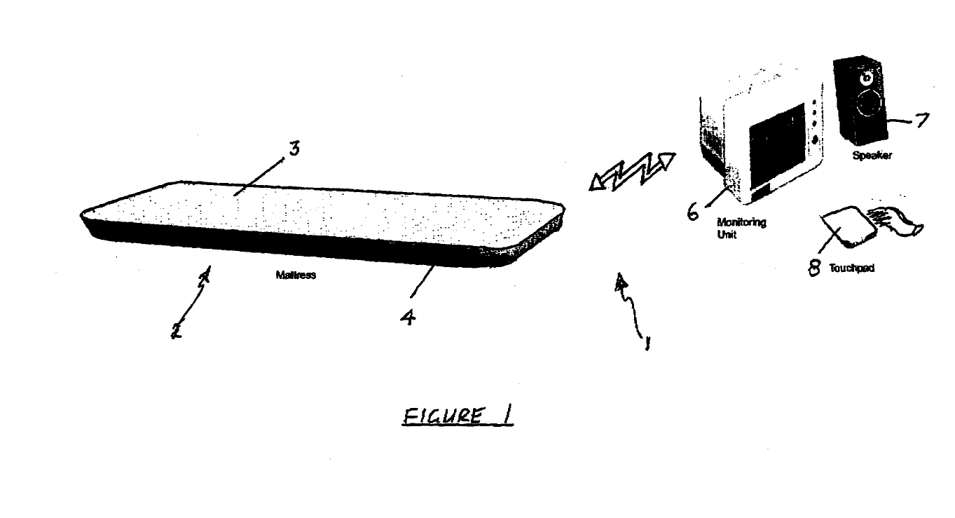

FIG. 1 shows a schematic layout of a sensing apparatus including sensor fields according to one embodiment

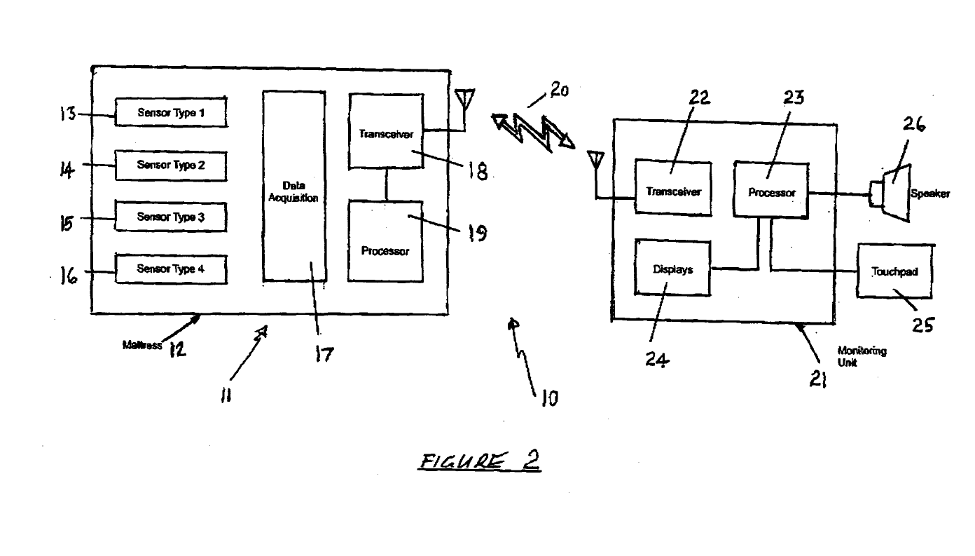

FIG. 2 shows a block diagram schematic layout of the assembly according to one embodiment.

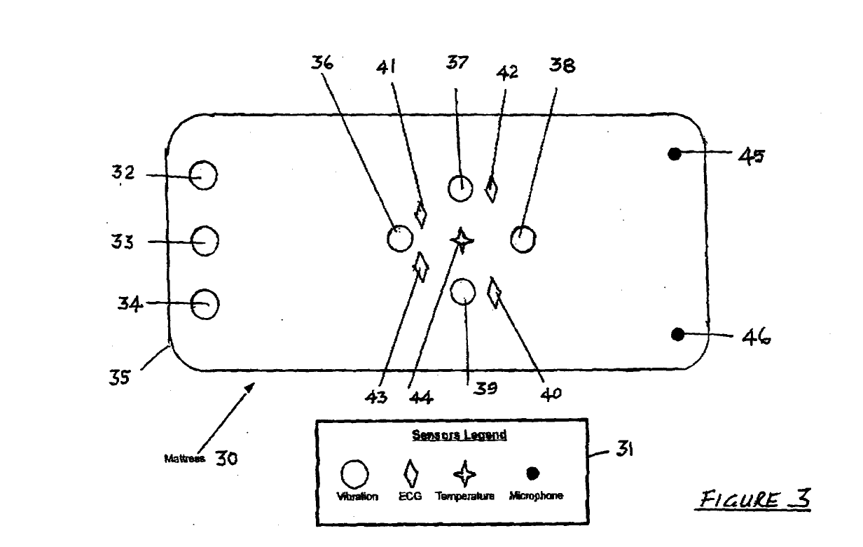

FIG. 3 shows a sensor field layout and sensor type legend.

### DETAILED DESCRIPTION

An example of the apparatus and method aspects of the invention will now be described according to various embodiments. A reference to simulated physical examination can be taken throughout as a reference to the ability of a practitioner using the sense of touch to remotely evaluate data obtained from a subject, using the apparatus of the present invention and preferably vibrational and/or temperature data in addition to or separate from visual and audio traces and to diagnose the condition of a subject based on using touch only; sight, sound and touch or sound and touch.

Referring to FIG. 1, there is shown an assembly 1 according to one embodiment Assembly 1 comprises detection station 2 including a surface 3 on which a subject/patient lies or sits. The surface 3 may according to a preferred embodiment be provided by a device 4 which can be a mat or mattress, located on a bed or chair, cot, table or like apparatus. A series of sensors arc located on or integrated in the device 4 to form sensor fields (see FIG. 3). Once the data is sensed, it is fed via a controller system including a transmitter and receiver to a processing station 5. Signals may be transmitted wirelessly or via land line to signal processing station 5. The transmitter delivers signals to a receiver which communicates with a processor. Signal outputs appear on monitor 6, separate speaker 7 or touch pad 8. Touch pad 8 enables an operator to receive signals by touch such as a vibration. Alternative devices which allow touch may be used such as a vibrating speaker, a temperature responsive device which is capable of reading thermal signals. An operator can elect which combination of outputs to analyse. For example the operator can elect to use the touch devices alone, the touch devices in conjunction with visual readouts or audio sounds. Use of touch allows a practitioner to simulate actual touch such as in the case of an examination of a fetus heartbeat.

Referring to FIG. 2, there is shown a schematic layout of a sensing and monitoring apparatus 10 according to one embodiment. Apparatus 10 comprises detection station 11 including a device 12 which is a mat, mattress, chair, tale or the like with which a subject engages for monitoring of that subject. Associated with device 12 are sensor fields or sensor types forming sensor fields. Shown arc four sensor types 13, 14, 15 and 16 which may comprise sensors for monitoring particular events such as but not limited to vibration, ECG, temperature. Sensor fields are established according to test requirements. Thus a sensor field may comprise one sensor or a cluster of like or different sensor types according to the type of event being monitored. Device 12 also has associated with it means 17 for data acquisition, a transceiver 18 and processor 19. Data collected is delivered by hard wire or wirelessly indicated by arrow 20 to a monitoring unit 21. Monitoring unit 21 includes a transceiver 22 and a processor 23 which delivers signal data to data outputs. One data output is display monitor 24. Other output devices comprise a touch pad 25 and speaker 26.

Once the data is sensed, it is fed via transceiver 18 to monitoring unit 21 for processing by processor 23. Signals may be transmitted wirelessly or via land line to unit 21. Transceivers and processors act as controllers to regulates signal transmission and processing Once signals are delivered to unit 21 the signals can be optimised by either auto analysis or manually by an operator with reference to signal outputs. Signal outputs appear on monitor 24, separate speaker 26 or touch pad 25. Touch pad 25 enables an operator to receive signals by touch such as a vibration. Alternative devices which allow touch may be used such as a vibrating speaker, a temperature responsive device which is capable of reading thermal signals. An operator can elect which combination of outputs to analyse. For example the operator can elect to use the touch devices alone, the touch (e.g. vibrations and temperature data) devices in conjunction with visual readouts or audio sounds. Use of touch allows a practitioner to simulate actual touch such as in the case of an examination of a fetus heartbeat. The signals are first optimized then analysed. According to one embodiment the data collected for analysis from the sensor fields may be transmitted for remote monitoring and analysis.

FIG. 3 shows a sensor field layout on a sensor mattress 30. Legend 31 shows examples of sensor types which can be employed to form sensor fields. Mattress 30 includes vibration sensors 32, 33 and 34 located at end 35 which forms one sensor field. Located centrally of the mattress 30 to form additional fields are vibration sensors 36, 37, 38 and 39 combined with ecg sensors 40, 41, 42 and 43 and temperature sensor 44. Sensor fields can be formed by one sensor or a series of sensors grouped close together or by a collection of spaced apart sensors either in direct contact with or not in contact with but proximate the subject. A series of sensors can be integrated into mattress 30 or attached to it. The sensor fields are arranged according to the required parameter/s of the subject to be measured. Sensors may be embedded or surface mounted so that their location is optimized relative to a subject providing a source of parameter/s which each sensor is capable of receiving. Each sensor in different locations can read at least one parameter or one sensor can read a variety of parameters emanating form different sites on the subject. In the example shown in figure 3 the sensor fields may be created by using the same type of sensors throughout the field or using a variety of different sensors to monitor different parameters. For Instance the sensor field may be created by a blend of microphones 45, 46 and sensors which can read parameters including vibrations, temperatures, pressures and electromagnetic fields.

Wireless sensors allow more comfortable subject friendly long term monitoring of pathological events which may occur intermittently or have a continually changing severity. In addition sensors of this type are safer to use during unattended studies at home with infants who are at risk when using alternative sensor systems requiring wired leads. It is a further object of the invention to provide a system of auto tuning sensors which are capable of detecting, measuring and analysing reduced states of consciousness. It is a further object of the invention to provide high sensitive monitoring sensors which allow differentiation of patient parameters and behaviour and which allow optimisation of signal data. . According to one embodiment, the sensors measure a time series of events. This enables analysis of the event time sequence, time distribution, individual event longevity, event time interrelationship with other events and changes occurring within or between recording sessions therein related to pathologies or treatments or other effects.

The present invention can be employed according to one embodiment for generating data which enables a method for classifying detected movements and breath sounds for the purpose of identifying and optimizing quiescent period and movement measures as an Indicator of sleep status and analysing pathological events. Prior art methods do not use a combination of body movement and breath sound signals to assess sleep status and identify events that arc affecting sleep and the interrelationship between both. It is known that movement is a normal part of the healthy sleep process. Also it is known that for sleep to have a restorative effect on a subject's conscious state a significant portion of their periods of quiescent sleep needs to be more than several minutes long. This method enables assessment of the quality and quantity of a subject's sleep and die affects of any pathological events or treatments on the subjects sleep by evaluating the time distribution and longevity of periods of movement and quiescence. For instance the presence of pathologies such as asthma or congestive heart failure or obstructive pulmonary disease (COPD) may affect a subject's sleep. The invention enables assessment of the subject's sleep quality as well as any effects of treatment of pathology as well as determining cause effect relationships associating pathologies such as apnoea, crepitations, lung infection, asthma, bronchitis, bronchopneumonia, lobar collapse, pleural effusion, pleurisy, pulmonary infarction and embolus with awakenings, disrupted sleep and or potential sleep loss.

For instance these methods also enable assessment of the frequency of changes in position of children due to movement occurring during sleep before and after adenotonsillectomy treatment for obstructive apnoea or other significant airway flow restriction. In this case treatment has been shown to reduce the number of periods of movement thereby increasing the periods of quiescence (ref: Choi et al: AM J. Rhinol Allergy 23, e56-e58, 2009). Alternatively, it enables the optimal titration of pharmaceutical treatments for conditions such as pain where too little treatment may result in reduced sleep reflected by increased movement due to excessive pain and too much drug treatment with strong pain killers may result in over sedation of the central respiratory drive system resulting in reduced respiration, central apnoeas, reduced body movement and possible death. The invention further enables the validation of the presence of the subject within sensing range of a sensor and also the proper function of the sensor. In particular some signals of interest such as breath sounds may stop during events such as central or obstructive apnea. Additionally breathing effort will cease during episodes of central apnea. The detection of the cessation for instance of breath sounds or breathing effort Is important for proper patient diagnosis. The invention includes use of sensors which can deliver data which is presented as vibrational signals to measure Including by use of the sense of touch, a broad range of biological signals related to breathing, heart, gut and other biological events. The validation of a target signal events cessation or significant reduction in magnitude is made using the evidence of detection from the same or close proximity sensor of one or more non target validation biological signal events such as heart beat or other events known to be occurring during the test period. This validation can be made manually or by using algorithms or other automated means to detect the presence of the validation signals and also detect the reduction or absence of the target signal. In addition presence of the target signal may be detected in the period just prior to and after its reduction or cessation.

The invention enables optimizing of data readings taken from one or more sensors which might be sensing the same parameters and then identifying the physiological event associated with this reading. The present invention further provides a method of optimizing data related to a series of measurements during a time sequence and length of movement period. According to one embodiment, remote wireless/contactless sensors arc located in a mattress or equivalent object such as a mat. Such sensors are also adaptable to a chair, bed, cot, operating table and the like. To achieve the most optimal results the sensors, preferably are placed in a mattress or mat, seats, cots, beds and the like and distributed over a predetermined area related to the body parameters required for measurement. The invention also has applications in other environments such as, but not limited to, astronaut condition monitoring, surgical patient monitoring in operating theatres and post operative wards, dental surgery applications, telemedicine or veterinary applications. The use of touch and the capacity to optimise signals obtained from contactless sensors in a variety of sensor fields may also be adapted for use in measurements in obstetric patients, monitoring of fetuses, in pediatric analyses, sports medicine and science and in corresponding veterinary applications. Sensors are positioned to enable readings of different parameters or multiple readings of the same body parameters. The system allows for the subject to move freely within the sensor field/s created by the one or more wireless sensors or a combination of wireless and contact sensors and a mat. At a given time during a recording period the signal obtained on one sensor for a parameter of interest may be better than the signal recorded for the same parameter via another sensor. The invention provides for optimising signals from one or more sensors sensing the same parameters once the data is obtained.

A first sensor might at one time point during a study provide data on optimal breathing effort or movement records representing a subjects inspiratory and expiratory efforts. A second sensor may provide the subjects optimal breathing sound records. As the study progresses the sensor provides the optimal signal to assess these parameters may change. The sensor providing the optimal signal is identifiable for the parameter of interest throughout a study where the optimal sensor may continually change during a study, Similarly the invention may also identify the optimal signal for breathing symptomatic of heart failure, asthma and other respiratory diseases such as COPD, pneumonia, pleurisy or lung infarction or pulmonary hypertension.

Following identification of the sensor providing the optimal signal or signals of interest, these signals can then be evaluated either in real time or retrospectively manually by an observer or in an automated fashion by using algorithms or filters or other means for instance to detect and assess signal events of interest. Sensors may be detecting more than one signal type such as when using vibration sensors to detect breath sounds and movements where many signals are aliased. A subject's temperature can be felt remotely by transmission of temperature data and using a heat/temperature detection device to allow a practitioner to feel the subject's temperature as if the subject was with the practitioner. This also allies to vibrational signal data. In this case after choosing the optimal sensor or sensors for signal detection at a given time point the aliased signals can be separated and each individual signal type of interest evaluated independently either by an observer or automatically. For instance a sound signal from one or more optimal sensors can be separated on the basis of the frequency signatures of specific sound types such as snore, normal breathing and heart sounds. These different signals can then be recorded and may be displayed as separated traces on a monitor enabling an observer to play back the trace while listening to the optimized separated signal of interest. Alternatively the separated signal may be automatically evaluated in real time or using a recorded record. Additionally detected signals may be output and observed or evaluated in a range of formats related to different sensory mechanisms as described herein such a visual, auditory or touch outputs.

The invention allows detection and analysis of a biological signal or signals of interest from a subject in a sensor field using one or more optimized sensors from the sensor field and then using automated, semi automated or non automated means to identify biological signatures of events of interest. The automated or semi automated means include but are not limited to filters, algorithms and comparators. Identification of the events of interest may require evaluation of one or more optimized signals from one or more optimized sensors. For instance, in the case of paradoxical breathing, different sites along the thorax such as the abdomen and chest may expand and contract with differing intensity and in differing phases. Breathing effort signals taken from different sensors in differing positions with respect to the thorax can detect these affects and help differentiate between paradoxical and normal breathing. In other examples signals from more than one sensor can help determine the site of a localised movement such as an arm or leg movement or the origin of a snore or cough sound which could be originating from the subject or the subjects partner. These determinations can be made by automated or non automated means. In other cases the methodology involves single target signals of interest such as breath or heart sounds and the detection of signal signatures which identify specific events of interest such as breathing related pathologies, heart related pathologies or a respiratory system response to a pathology or therapeutic treatment or normal physiological event eg—detection of wheezing, bruxing or breathing or heart signal changes during the REM state or the respiratory depression occurring as a result of opioid ingestion.

The apparatus and method enables detection of breathing flow and breathing effort related signals. These signals can be used for assessment of a range of conditions including central or obstructive origin sleep apnea or hypoventilation eg—a hypopnea. Breathing flow and effort related signals are detected by sensors in a sensor field and the optimal signals identified as described. An additional advantage enabled by monitoring the change with time in the optimal sensor to be used for monitoring a particular signal event relates to measurement of the time sequence of subject movement which is an important aspect related to measuring sleep quality and the effect of pathologies or treatments on it. Gross body movements made by the subject are often reflected in changes in the position in the “sensor field” of the optimal sensor required for monitoring signals of interest The frequency of change in the optimal sensor is a measure of the frequency of positional change of the subject which as previously mentioned is an important indicator of sleep pathology for instance in children requiring adenotonsillectomy.

An advantage of contactless monitoring sensors is that a subject does not have to lie or sit against the sensors and they can detect extra sound and movement and associated events. In the prior art, systems relied on signals and sensors which were attached to the subject. According to the invention a wireless system provides significant advantages. The presence and quality of sleep can be determined from the magnitude and nature of body movements and the use of breathing sounds. Analysis of breath sounds can indicate whether the sounds represent a subject in the asleep or the awake state.

The apparatus and method also enables targeting of physiological signals of interest to be normalized so that the magnitude of a signal taken at one time point can be compared with one taken at another time point during a study or during different studies. The magnitude of a biological signal sensed by a non-contact sensor will vary significantly as a subject moves in relationship to the sensor. In prior art systems involving attached sensors this variation is not present since the spatial relationship between the subject and sensor remains constant. The magnitude of a target signal of interest such as a pathological signal like a snore or asthma or COPD sound represents one estimate of its severity. This magnitude can vary during a test period. Other non pathological sounds such as normal breath or heart sounds will generally maintain their magnitude throughout a study period.

The apparatus and method also allows assessment of the severity of pathological vibrational signals obtained from either sensors in contact with a subject, sensors not in direct contact with a subject, such as a signal of upper airway flow resistance, cough, snore, asthma or COPD or crepitation or tooth grinding or esophageal reflux severity. The method involves measuring the intensity or power or energy of a signal or portion of a signal of interest. In the prior art the severity of pathologies such as snore, wheeze and upper airway flow resistance tend to be measured in the metrics of event occurrence frequency. According to one embodiment, “contact” sensors in combination with a wireless sensor field may be employed to obtain the required signals. In particular, some signals of interest such as oximetry involve use of sensors which to date still require attachment to the subject. It is envisaged that these sensor types may be used with the apparatus and their data output combined with those of the signals.

According to one embodiment, a whole bedding mattress can be adapted as a sensor mattress and pick up breathing vibrations and breath sounds duration activities such as snoring. Use of wireless sensor amplifiers monitor displays and recorders allows a method for continuous physical examination either in real time or retrospectively. No leads are used so there is no prospect of the patient or subject being fouled by leads. This is particularly advantageous for subjects when monitored at home. Sensors pick up low frequency movement and high frequency is sound. Sensors are preferably integrated into the mattress along with one or more microphones. Placing microphones at the top corners is desirable as this is least likely to be covered by sheets or blankets.

Strategic orientation of room and proximity microphones and their relativity to sensors allow optimal parameter detection. For example, comparing the sounds captured from the mat sensors and the room sounds picked up by microphones or sound sensors, identification can be made of restricted breathing. Measurement of sound intensity can also be an indicator or damage to tissues such as carotid artery and measure of breathing effort.

An advantage of the present invention is that it allows identification of sounds and pathologies that the prior art methodologies have not been able to adequately determine. The wireless monitoring system is very sensitive and is responsive to subtle sounds. The sensitivity allows the system to monitor length of movements and occurrence of types of certain movements such as REM movements. Those parameters along with evaluation of breathing effort and breath sound parameters as a metric of air flow will identify occurrences of sleep apnoea or hypopnea. The method provides a means of assessing sleep and events occurring during sleep by using a mat as part of the sensor field for getting data. In the prior art an EEG was used which includes sensors which arc in direct contact with the subject's body. According to the one embodiment, EEG, EOG, EMG sensors may be placed in or about the mat including potentially within a pillow.

According to one embodiment of the method aspect, the contactless sensors detect flow restriction which enables a determination of the power or energy involved in this restriction which may present as snoring and may be indicative of or a risk factor for physiological damage such as mechanical damage to the carotid artery. One can consider the severity of flow restriction or snoring or the severity of the frequency of snoring or the rise in amplitude of breath sounds and high frequency inspiratory or expiratory sounds and draw conclusions from that study. Stroke damage may for instance be related to upper airway flow resistance, snoring and sleep apnea. Vibrational breathing related energy may transmit mechanical effects on proximate soft tissues which might suggest a relationship between flow restriction and other conditions such as stroke, and arterial disease (see references).

The apparatus and method can be used to assist in determining the site and cause of flow restriction being recorded. Breath sounds can be recorded both during sleep and the awake state. Some flow restriction related sounds such as snoring will only occur during sleep due to alterations in jaw position and muscle tone. In other cases wheeze sounds may be asthma related or sleep related. Asthma related wheeze will persist both during sleep and wake whereas sleep related wheeze sounds will disappear during wake. Wheeze sounds of each type can be difficult to differentiate. The invention enables differentiation. Other flow restriction related sounds originating in the upper airway have different signatures dependent on the site of origin. The site of origin can be determined through evaluation of the signal frequency signatures and position in the breathing cycle.

According to a preferred embodiment, multiple sensors arc selected for optimization. For example, a vibrational signal from one sensor is compared to the same parameter signal from another sensor. If one has a better vibrational signal but which is affected by noise, compared to a much weaker vibrational signal unaffected by noise the worst signal can be identified and its characteristics used to separate the noise from the stronger vibrational signal thereby providing an optimal signal from those vibrational signals available. In this way, signal quality comparisons may be made for all required parameters to determine optima) signals on which to base a diagnosis.

According to one embodiment there is provided a means for selecting optimal parameter signals from the at least one sensor. Preferably the optimization of signals may either be done manually by looking at the display or automatically without a need for a display using an algorithm.

According to one embodiment a display is used for signal optimization. According to an alternative embodiment, an automated algorithm is used to analyse output data without a visual display.

According to an alternative embodiment, algorithms are used to analyse the above periods of waking and sleeping for the particular subject Once periods are identified, the signals are also used to identify various potentially related events including REM sleep, movements, sighing, irregular breathing, cardiac function, respiratory function and other events which may affect sleep quality to enable diagnosis.

According to one embodiment of the above system the speaker cone of a broad frequency range audio speaker which can also output sub audible signals can be used to feel vibrations as they occur and are viewed on the visual trace. This can be particularly useful for the observer where signals such as fetal heart sounds have low power and can be hard to hear. Alternatively additional information can be obtained feeling vibrations where their frequency is below the normal audible range. This embodiment is useful for clinicians who are trained to use palpation as part of their normal method of diagnosis. For instance the means to feel a signal can be useful in the case of a bruit such as a cardiac shunt. In the case of some low frequency sounds the observer can also see the movement in the speaker cone as well as feeling it whereas the sound may be difficult to detect by hearing. In another embodiment vibrations can be output through objects other than a speaker such as a “pad” against which the observer rests their hand.

The invention provides for the detection of biological signals using one or more contact sensors attached to the subject or sensors in a sensor field and the output of one or more signals of interest to an observer using a signal output means involving feel and sound and or touch. By this means an observer can use two or more senses to evaluate biological signals of interest. In some cases a single signal such as related to a heart movement can be observed using more than one sense by feeling the heart movement via a speaker cone or touch “pad”, while looking at a visual trace representing the heart movement and hearing the heart sounds. In other cases multiple signals may be reviewed at once with one being touch reviewed while others are observed via visual trace and or listened to. Alternatively the visual output can be made for instance via a simulated physical or computer generated model of the subject In this case signals can be more readily understood particularly by less specialized observers by showing a physical or simulated recreation or representation of the signals source and characteristics.

According to one embodiment the above system may be used in conjunction with EEG, ECG, video, temperature or movement monitors to record additional signals. Preferably the readout device is a computer screen display where traces of different channels are displayed one below the other to present the signals synchronized in time. An electronic processor may be employed to analyse the recorded information and identify parts of the traces using algorithms.

The invention further provides a method and apparatus for analyzing the occurrence of individual events as well as relationships between a range of biological and environmental signals occurring during testing of a subject placed within a sensor field. During a test period often a number of single events of interest as well as cause and affect events will occur. The method aspect of the invention enables the detection and magnitude quantification of a range of biological and environmental signals during a testing period of a subject. In many cases a second biological event will occur as a result of one or more prior occurring events which may be of biological or environmental origin. The secondary event may be a direct sign of pathology such as abnormal sleep quality or heart rhythm affects. For example a subject with obstructive sleep apnoea will have a sleep apnoea event which will often be followed by a sleep arousal response. In another example a sleeping subject in an environment which is noisy due to such noise influences as television, radio or human conversion or airplane noise may have sleep arousals caused by the prior occurring environmental noises. A subject with nocturnal asthma may have wheeze or coughing instances which cause subsequent arousal of the subject from deep sleep. In another case a subject with both sleep apnoea and asthma may have arousals due to one or both of these potentially causal events. In other cases pathological events such as asthma wheeze which can potentially affect sleep quality may have no such affect in some subjects. In another example a sleep apnoea event may have a causal relationship with a following heart arrhythmia occurrence. In order to properly treat subjects and assess the efficacy of treatment it is important to understand which biological or environmental events are correlated and which are independent. The invention enables the analyzer to pick one or more events of interest using either a library or an algorithm to search for like signals in the record. Alternatively the person analyzing a record can manually identify and choose a signal of interest by listening to or looking at the visual display or using touch to identify one or more examples of the signal. The apparatus and method enables the analysis of the characteristics of these examples and also can automatically search for all similar events occurrences during the test period. The signal characteristics can include signal frequency patterns; wave form patterns; signal magnitudes; relationship to inspiratory and expiratory cycles and other signal signature characteristics. Following identification of individual events of interest then the invention enables the automated identification and quantification of two or more biological or environmental events which may be related. For instance a subjects recording period can be assessed for all instances involving a wheeze event preceding a sleep arousal event by less than a specified time period, for instance less than five seconds or two breath cycles. A series of logical search relationships can thus be set up or programmed in order to quickly analyse a complete recording record. The analysis can also quantify the number of combined events of interest occurring during the test period. In another example an external environmental sound can be identified and then all cases where arousals may be caused by this external noise identified. In this case the logical search relationship would involve a programmed search algorithm developed to look for cases where sleep arousal follows instances of the environmental sound occurring for instance within a few seconds or two breath cycles. This analysis may then be used to separate affects on the patients sleep by pathologies from those caused by environments affects. In another example all cases where heart arrhythmias arc occurring as a result of sleep apnoea and lowered blood oxygen levels may be identified. It will be realized that in other cases two or more events can be analyzed to determine on how many occasions they occur where a predefined time or causal relationship does not exist. The invention thus enables one or more events of interest to be identified, logical search relationships to be defined either as a standard tool or by the person analyzing the record and then for the automated searching, identification and analysis of all such instances or relationships occurring during the record period. The invention thereby provides a simplified way of identifying an event or group of events of interest and then finding all similar cases during a current or previous record.

Throughout the specification a reference to sensors can be taken to be a reference to detection sensors which are either connected to a subject or not positively connected to a subject from which sensed data is taken. A subject may have transient contact with a non contact sensor in a sensor field during a reading period, but the sensors are not fixed to the subject but rather fixed to a location (embedded or surface mounted), which together with other sensors defines a field in which the subject is free to move. In this way sensors can detect parameter data by transient subject contact or remotely from the location on or in the subject from where data is read.

Sensor Field Optimisation

There are significant advantages in the use of contactless sensors for sensing parameters of a subject. There are also disadvantages in use of sensors which are physically attached to and move with the subject. Measurements from those sensors may be compromised when a subject lies on or dislodges the sensor. Children in particular are prone to unwanted removal of sensors by movements during sleep. The creation of a sensor field or fields in which sensors are remote from the subject, avoids the disadvantages of contact sensors. However, when sensors are not in direct contact with a selected site on a subject, more than one sensor can sense one or more parameters relating to the subject In that case signals can be optimised to ascertain the best read or signal quality from a variety of read outs for one sensor as they relate to one parameter.

In the sensor field system, patients move around during the night on a mat or mattress which housed multiple sensors. It was assumed that the highest quality signals would be provided by one sensor for all biological signs of interest and that all of these would be available from that one sensor at a given time point. It was also assumed that good signal records may not be available at some times during the test period due to the subject's position, bed clothes or other factors creating sensor interference. It was initially thought that highly accurate quantitative assessments of a subject's status could not be made with respect to sleep disordered breathing severity or severity of other pathologies. Although it would be possible to do qualitative assessments of pathology incidence, it was not clear whether quantitatively accurate assessments could be made which would enable optimal therapeutic prescription.

Two unexpected results related to the invention were as follows:

1 the optimal version of signals of different types were found to be not always all available from the same sensor as discussed below. This presented an unexpected complexity when trying to understand and use records.

2 In spite of this complexity it was found that by developing a novel method of optimizing signals from the sensor field it was possible to achieve very accurate quantitative results when compared to the industry standard and much more invasive technologies such as Polysomnography (PSG).

Studies comparing results of PSG and Sensor field quantitative assessments for the same patient achieved very accurate and equivalent results. This enabled the inventors to achieve of an optimal therapeutic prescription without using PSG and with an expectation of accurate results.

Signals detected using a sensor field apparatus can now be used for a wide variety of purposes. When a signal of interest is being studied on one or more sensors at the same time in a sensor field, the methods used to analyze signal events will vary depending on the signal type and the requirements of the analysis. As a test subject moves within a sensor field, the signal(s) of interest may be best identified and studied on different sensors at different times throughout the study period. In addition if several signal types are being studied concurrently during a test period, at any time point different sensors may be detecting the best record of different signal types. For instance at one moment one sensor may detect the best signal for breath sounds, another for heart sounds and another for breathing effort movements. Alternatively during another period all parameter signals may be best detected on the same sensor.

In order complete analysis of each signal type detected in the sensor field a method of signal optimization is used throughout the test period in order to identify the best signal records for use in the analysis. The optimization process varies dependent on the analysis requirements.

### Example I

Signal optimizing in the following circumstances;
(1) Analysis: Determining the presence of partially obstructed breathing (POB) during a test period
Optimisation Process;
   (i) Evaluate records from all sensors for the presence of Partially Obstructed Breathing (POB) for the entire test period . Using filters, search for the presence of signals above background noise in the required frequency domains (see definition of POB)
   (ii) Identify the sequential time periods throughout the test period within which POB signals are detected on one or more sensors at the same time.
(2) Analysis: Determine the presence or absence of breathing flow periods occurring during the test period for apnea assessment
Optimisation Process
   (i) Throughout the period evaluate all sensor records for the presence of breathing flow related signals. Using filters search for the presence of breathing flow signals above background noise within the time increments of a breathing cycle (i.e. breathing effort frequency and breathing flow frequency) domains (may be normal breathing or SDB).
   (ii) Identify periods where no breathing flow signal occurs on any sensor but where any other biological signal from the subject is present at the same time on one or more of the sensors. (use filters to detect any biological signals above noise other than breathing sound signals).
(3) Analysis: Generate and or store a single contiguous record of the patients breathing flow signal (e.g. for use in recording and then replaying the signal on one channel in order to simplify the clinical review process. This allows a clinician to listen to one record as though it had been detected via a single sensor or stethoscope)
Optimisation Process
   (i) Evaluate all sensor records throughout the test period for the presence of breathing flow related signals. Using filters search for the presence of signals above background noise in the required time (i.e. breathing effort frequency) and frequency (i.e. breathing flow frequency) domains (may be normal breathing or SDB).
   (ii) During each time period sequentially throughout the test period identify which sensor(s) is detecting a breathing flow related signal and if detected on more than one sensor which signal from which sensor has the highest quality (for Instance highest signal to noise ratio)
   (iii) During each time period throughout the test period output via or copy and or record the highest quality version of the subjects breathing flow related signal to a single channel.

In practice, a patient under monitoring can move to and away from contactless sensors but the sensors provide a high quality signal from the patient into the relevant sensor/s. Multiple vibrational or electromagnetic or other signals can be read by multiple sensors creating a sensor field, following which the group of signals may be optimized to provide the best quality signal dependent upon the diagnostic objective to be achieved by the selected signals. For each sensor an accompanying microphone is provided to sense subject's own and environmental sounds so that unwanted noise can be explained and ignored or used in assisting diagnosis. For example a signal or set of signals can be optimized for the purpose of diagnosing conditions which may be related to effects of vibrational energy in a subject. Alternatively, the signals may be optimized for the purpose of diagnosing apnea using breath sounds and movements. Typical parameters picked up by the monitors include breathing movements, air flow, vibrations, gut noises, heart beats, body movements, wheezing, coughing, mouth movements, blood flows, fetal arousal, hiccups. Each parameter can be monitored/detected by potentially each sensor with each providing a trace for one or more selected parameters which can then be optimized to assist diagnosis. Optimizing can be taken to mean a process whereby a practitioner skilled in the art is able to review ail signal results from all sensors and select those signals which are best for diagnostic purposes. This process may alternatively be completed by an automated means. For example signals could be taken from three sensors to reduce noise and increase the quality of the wanted aspects of the signal for diagnostic purposes. This may be performed by for Instance identifying environmental noise (such as a partner sleeping, external or room noise) in one sensor and subtracting that from the same noise in another sensor which may have higher quality reading of the wanted parameters. The result is the highest quality clean signal free of unwanted noise data.

According to a method aspect, signals from each sensor can be used to interpret the presence or absence of vibrational signature signals that may be related to events of diagnostic interest for a subject. A signal signature of interest for diagnostic purposes can be used to determine the origin of a causative event whether environmental, physiological or atmospheric. Once signals are optimized from the plurality of available sensors and the data they collect, the practitioner can use the optimized signals to study events which may have generated such signals. Events could include apnea, hypopnea—central and obstructive, heart pathologies such as murmurs, breathing flow restriction as it relates to the healthy sleep wake cycle, asthma, crepitation, fetal movement, fetal breathing effort or fetal blood flow related sounds such as placental blood flow sounds. The optimized signals may also be used to analyse breathing patterns related to the air flow patterns in the selected signal to determine using time, frequencies, power, wavelengths and amplitudes any airway restrictions from snoring, wheezing. Certain pathologies can be shown from certain wave patterns. As an example, wheezing can be determined by reference to specific waveforms as with snoring which can create frequencies of up to 250 Hz. Obstructive breathing may not have a recognizable wave form but may have mixed leaks and demonstrate frequencies around the range 250-500 Hz. By studying signal power and frequency, time and frequency, amplitude and time a practitioner can determine a diagnosis. Other pathologies can be assessed adopting a similar approach to signal optimizing for determination of particular events. Although the invention has been described with reference to a particular example it should be appreciated that many other options are available. One of the major advantages of this invention is that it is not necessary for the medical staff to continuously observe the patient. In fact a patient with sleep apnoea may be left overnight and monitored using the system.

The traces recorded over a period of say eight hours may then be reviewed very quickly the following day by fast forwarding them or by identifying particular occurrences of activity which require closer monitoring by the medical staff. These can be identified automatically by algorithms acting on the traces, or they could be identified during fast forward scrolling of the traces by a suitably qualified clinician. For instance fast forward monitoring of the visual trace could identify particular activity which could then be investigated further. On the other hand the monitoring could discount otherwise unusual activity occurring in the traces, for instance environmental noise or coughing etc. It should be appreciated that all signals can be transmitted from remote locations to a central monitoring station.

A signal of interest to an observer can be replayed involving providing a visual signal trace, a tactile signal (via a speaker or the like—see FIG. 1) and (where relevant) a sound signal (i.e. some signals may be too low frequency to hear).

In another example of methods of using signals from one or more sensors, the operator
identities origin site and cause of signal from a signal signature, inclusion frequency information (eg trachea, nasal, lung)
ascertains a presence/absence of signal in sleep/wake (eg wheeze from Asthma v upper airway)
determination of paradoxical breathing using two or more sensors
identify site/s of gross body movement (leg v arm movement); and
ascertains origin of sounds (subject v partner snore, atmospheric sounds etc)

Manual or automated methods can be used including algorithms to detect pathologies, effects of treatments or biological responses to or during events (eg breathing, heart or other related affects). The methodology allows for the analysis sleep quality and quantity by measuring periods of movement and quiescence, thereby enabling the practitioner to look at pathologies or treatments and their affect on the subjects sleep quality/quantity

The method also allows a means for measuring foetal and maternal health using a wireless sensing system to make measures including foetal movements and/or umbilical and/or placental and/or foetal blood flow.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. An assembly for monitoring physiological parameters of a living subject at a sensing station, the assembly comprising :
at least one sensor field comprising sensors capable of sensing one or more said parameters of the subject in each said at least one sensor fields;
electronic transmitter for transmitting data sensed by said sensors of said at least one sensor field to at least one data processing station;
wherein the data comprises information regarding sensed movements and sounds within the range of 0.5 Hz-20,000 Hz; and
wherein data obtained from the sensor fields comprises data outputted as information capable of interpretation using the sense of touch.

2-15. (canceled)

16. An assembly according to claim 1 wherein said data is obtained from the subject in real time.

17. An assembly according to claim 1 wherein said data is optimized and analyzed.

18. An assembly according to claim 1 wherein, said data is obtained via said sensors from parameters selected for data measurement from a list consisting of breathing (2-3 kHz), snoring (200-900 Hz), heartbeat, body movements, body position, gut activity, reflux, jaw movements, sleep efficiency, sleep performance including snoring, sleep apnea, restricted airways, asthma, period spent asleep or awake, fetal heart beat, fetal movements, placental blood flow and the like; sounds and vibrations; blood flow in various blood vessels and organs, digestion and skeletal muscle activation and joint movement vibrations; pneumonia, pleural rub snoring, crepitations, wheezing, and any combination thereof.

19. An assembly according to claim 16 wherein, pathologies of a subject I are detectable through analysis of data signals which include signals related to pathalogy selected from a list consisting of; hypopnea, obstructed breathing, snoring, coughing, wheezing, crepitation, tooth grinding, vomiting, body movements such as myoclonus and esophageal reflux severity, esophageal manometry to measure flow resistance, esophageal reflux catheter to measure reflux severity, actigraphy, muscle activity EMG, electroencephalograms EEG, eye movement (EOG), electrocardiogram ECG, oximetry; vomiting, crying, groaning, sniffing, shivering, heart valve movement and sounds, fibrillations, ectopic beats, tremors, rolling movements, fitting, digestive sounds, bodily fluid flows, breathing effort, bruxism, fetal breathing effort, non diagnostic or not necessarily diagnostic environmental noise; temperature, sweating, biochemical or respiratory status and position in the sensor field; the REM state or the respiratory depression occurring as a result of opioid ingestion; mouth movements, blood flows, fetal arousal, hiccups, and any combination thereof.

20-31. (canceled)

32. A method of data collection from a living subject and analysis of said data using an assembly comprising,
a plurality of sensors;
at least one sensor field formed by at least one sensor from said plurality of sensors;
transmitter for transmitting data sensed by said sensors to at least one data processing station;
the method comprising the steps of:
locating a subject on or near a monitoring device;
arranging a plurality of sensors relative to the subject to create at least one sensor field about the subject the sensors being able to sense at least one parameter of said subject;
transmitting data measured from the sensors in sensor fields to a receiver; optimizing data signals received from the sensor fields by selecting optimal signals from the data,
wherein the data includes sensed low frequency movements and high frequency sound within the range of 0.5 Hz-20000 Hz;
and wherein data obtained from the sensor fields comprises data outputted as information capable of analysis by touch.

33. A method according to claim 32 further comprising the step of combining touch analysis with analysis using visual read outs and aural data.

34. A method according to claim 33 wherein the data capable of analysis by touch comprises vibrations relating to one or more parameters and temperature of the subject.

35-40. (canceled)

41. A method according to claim 31 wherein following selection of a parameter and a measurement duration period for the parameter, optimizing the measured signals comprises the steps of:
i) evaluating data measured from all sensors in all sensor fields to determine the presence of the parameter over the measurement duration period;
ii) determining the presence or absence of signals relative to threshold noise in required frequency domains;
iii) identifying sequential time periods throughout the test period within which the measured parameter or parameters signals are detected on one or more sensors at the same time;
iv) during each time period sequentially throughout the test period, identifying which sensor if any, is detecting the selected parameter.

42-43. (canceled)

44. A method according to claim 41 using an algorithm to automatically optimise a signal.

45. A method according to claim 44 further comprising the step of identifying if a subject has Central Sleep Apnea, Obstructive Sleep Apnea or Mixed Sleep Apnea, utilizing the aforesaid steps, and with reference to body movement sounds of breathing effort sounds.

46. (canceled)

47. A method according to claim 32, further comprising the step of selecting one or more said parameters for optimising, and wherein, the data signals fall within the Range 0.5 Hz-20,000 Hz.

48. A method according to claim 32 wherein the plurality of sensors comprise at least one sensor capable of sensing a parameter selected from a list consisting of breathing, heartbeat/activity, body movements, body position, gut activity, reflux, jaw movements, sleep efficiency, sleep performance including snoring, sleep apnea, restricted airways, asthma, period spent asleep or awake and fetal heart beat, fetal movements, placental blood flow and the like; sounds and vibrations; blood flow in various blood vessels and organs, digestion and skeletal muscle activation and joint movement vibrations; pneumonia, pleural rub snoring, crepitations and wheezing, pathologies including hypopnea, obstructed breathing, snoring, cough, wheeze, crepitation, tooth grinding, vomiting, body movements such as myoclonus and esophageal reflux severity, esophageal manometry to measure flow resistance, esophageal reflux catheter to measure reflux severity, actigraphy, muscle activity EMG, electroencephalograms EEG, eye movement (EOG), electrocardiogram ECG, oximetry; vomiting, crying, groaning, sniffing, shivering, heart valve movement and sounds, fibrillations, ectopic beats, tremors, rolling movements, fitting, digestive sounds, bodily fluid flows, breathing effort, bruxism, fetal breathing effort, non diagnostic or not necessarily diagnostic environmental noise; temperature, sweating, biochemical or respiratory status and position in the sensor field; the REM state or the respiratory depression occurring as a result of opioid ingestion; mouth movements, blood flows, fetal arousal, hiccups, and any combination thereof.

49. (canceled)
